(19) United States
(12) Patent Application Publication
(10) Pub. No.: US 2014/0329779 A1
(43) Pub. Date: Nov. 6, 2014

(54) Clear solid sticks with solubilized pharmaceutical agents

(71) Applicants: Leonard Mackles, New York, NY (US); William Bess, Edison, NJ (US)

(72) Inventors: Leonard Mackles, New York, NY (US); William Bess, Edison, NJ (US)

(73) Assignee: PRODUCT AND TECHNOLOGY PARTNERS LLC, Newark, NJ (US)

(21) Appl. No.: 14/234,609

(22) PCT Filed: Jan. 22, 2013
(86) PCT No.: PCT/US2013/022435

§ 371 Date: Jan. 23, 2014

Related U.S. Application Data
(63) Continuation of application No. 13/359,451, filed on Jan. 26, 2012, now abandoned.

(51) Int. Cl. A61K 9/06; A61K 31/19; A61K 31/616; A61K 31/192; A61K 31/60

(52) U.S. Cl. CPC A61K 9/06; A61K 31/192; A61K 31/60; A61K 31/616; A61K 31/19
USPC 514/159; 514/570; 514/165; 514/557

(57) ABSTRACT

The invention discloses a solid transparent vehicle utilizing DBS in which a topical pharmaceutical active or cosmetic active is solubilized without the use of a monohydric and/or polyhydric alcohol thus providing a non-tacky, clear solid, which prevents segregation and insures uniformity in dosing.

### FIELD OF THE INVENTION

Abradable clear solid sticks for topical delivery of pharmaceutically active substances

### BACKGROUND

The use of solid sticks to deliver active ingredients or cosmetics to the skin is well known in the art. Examples include suspended rubifacients, styptic sticks, lip balms, lipsticks, and deodorants. During manufacture, and potentially on storage, the active pharmaceutical may segregate and thus the product may not provide a uniform dose of the pharmaceutical active or cosmetic to the skin. Clear solids in which the pharmaceutical active is solubilized eliminate the possibility of segregation. Clear solids utilizing sodium stearate soaps are also well known in the art, however, these compositions have limitations that may affect the stability or efficacy of many pharmaceutical actives or the integrity of the solid vehicle itself. For example, acid based actives such as salicylic acid cannot be utilized in these compositions due to its low pH which will cause the solid vehicle to liquefy.

In recent years the development of clear antiperspirant solids has been disclosed in the art utilizing a dibenzylidene sorbitol (DBS) gelling agent to form the clear solid. The first of these compositions did not contain organic carbonates since they are not required to produce a clear solid. More recently antiperspirant solids utilize an organic carbonate, suitably propylene carbonate, as part of the composition in order to reduce the temperature required to manufacture composition. However, these sticks cannot be made clear until and unless a mono or polyhydric alcohol is included in the composition. A clear antiperspirant solid cannot be prepared without the use of a mono and/or polyhydric alcohol.

These solid sticks have found limited commercial use due to several factors, one of which is their tendency to be very tacky when used. This tackiness is caused in part by the inclusion of the monohydric and/or polyhydric alcohols, such as propylene glycol, which are required to achieve clarity in these solid sticks. This is due to the fact that antiperspirant compounds are not soluble in the DBS based vehicle unless the monohydric and/or polyhydric alcohol is added. In some instances the monohydric and/or polyhydric alcohol is complexed with the antiperspirant compound before inclusion in the final solid stick. An example of such a complex is Rehydrol®, an aluminum chlorohydrate-propylene glycol complex. In other instances, the monohydric and/or polyhydric alcohol is added as a separate component of the final stick composition.

### DISCUSSION OF THE PRIOR ART

U.S. Pat. No. 4,346,079 (Roehl) discloses a transparent antiperspirant solid utilizing dibenzylidene sorbitol, a monohydric alcohol, a polyhydric alcohol, a fatty acid alkanolamide, a propylene glycol-ethylene glycol condensate, and a metallic antiperspirant salt. The alcohol, polyhydric alcohol, alkanolamide, and glycol condensate are needed in order to solubilize the metallic antiperspirant salt and thus achieve transparency.

U.S. Pat. No. 4,722,835 (Schamper) discloses solid metallic salt antiperspirant compositions containing dibenzylidene sorbitol, propylene carbonate, a monohydroxy and/or polyhydroxy alcohol, a fatty acid, and a basic metallic salt. The alcohol is required to achieve the desired clarity of the composition.

U.S. Pat. No. 5,059,628 (Tsuda) discloses pharmaceutical gels containing dibenzylidene sorbitol, proline esters and dibenzylidene sorbitol. Optionally, mono and/or polyhydric alcohols may be utilized. The compositions disclosed are not transparent and require the use of the proline esters, which are used as penetration enhancers for topically applied pharmaceutical actives.

U.S. Pat. No. 6,881,776 (Butuc) discloses a combination of propylene carbonate and dibenzylidene sorbitol in which both compounds are utilized as solvents which are gelled by the further addition of polymeric compounds such as star polymers, triblock polymers, and others.

The prior art contains a number of patents and patent applications which encompass the use of dibenzylidene sorbitol as a gelling agent for pharmaceutical products. However, nowhere in the prior art is there mentioned nor anticipated the compositions described in this invention.

Diez et al. WO 2006014831 A1 teaches the use of polyols (described as any compound having two or more hydroxyl groups) combined with carbonates to form pharmaceutical compositions useful for topical application. This definition would include dibenzylidene sorbitol since it contains two hydroxyl groups. However, Diez states that “. . . the compositions of the present invention can be applied to wet skin due to the compatibility of the carbonates and polyols with water.” Dibenzylidene sorbitol is not compatible with water and thus it is clear that Diez is neither teaching nor anticipating the use of dibenzylidene sorbitol as a polyol in his invention. Furthermore Diez does not teach nor anticipate clear solids using dibenzylidene sorbitol.

Kneafsey et al WO 01/92430 A1 teaches the use of, dibenzylidene sorbitol to form gels and sticks containing solvents capable of solubilizing cyanoacrylate compounds. None of the compositions of the instant invention anticipate nor incorporate solvents capable of solubilizing cyanoacrylate compounds. The alkylene carbonates of the instant invention are not solubilizing agents for cyanoacrylate compounds thus Kneafsey does not teach nor anticipate the use of alkylene carbonates in combination with dibenzylidene sorbitol. Furthermore Kneafsey does not teach nor anticipate clear solids containing pharmaceutical active ingredients useful for topical application to the skin.

Mackles et al U.S. Pat. No. 5,587,152 teaches the use of dibenzylidene sorbitol to form sticks with a “water-insoluble ester of a water-soluble acid having solubility in water of greater than 50% w/w at 20° C.” The alkylene carbonates of the instant invention are not water insoluble esters of a water soluble acid having solubility in water of greater than 50% w/w at 20° C. Thus Mackles does not teach nor anticipate the use of alkylene carbonates in combination with dibenzylidene sorbitol.

Lahanas et al. U.S. Pat. No. 5,750,125 teaches the use of dibenzylidene sorbitol in pharmaceutical applications with solvents within a specific range of refractive index. The alkylene carbonates of the present invention do not fall within the specified refractive index range. Thus Lahanas does not anticipate nor teach the use of alkylene carbonates in combination with dibenzylidene sorbitol

Stoppolino U.S. Pat. No. 5,767,161 teaches the use of dibenzylidene sorbitol to form a solid stick containing ibuprofen. This stick contains a total weight percentage of 96% mono and polyhydric alcohols. Alkylene carbonates are not mentioned nor taught. Thus Stroppolino is not teaching nor anticipating the use of alkylene carbonates nor is he teaching nor anticipating the formation of solid sticks without the use of mono and/or polyhydric alcohols as part of the formulation.

### SUMMARY OF THE INVENTION

The instant invention discloses a solid transparent vehicle utilizing DBS (benzylidine sorbitol), in which a topical pharmaceutical active or cosmetic active is solubilized without the use of a monohydric and/or polyhydric alcohol other than benzylidine sorbitol, thus providing a non-tacky, clear solid, which prevents segregation and insures uniformity in dosing. The instant invention differs from all the prior art in that the use of monohydric and/or polyhydric alcohols is not required to achieve clarity.

The instant invention utilizes a combination of DBS and an organic carbonate for a hitherto undiscovered and undisclosed use, the formation of clear solids in which non-antiperspirant topical pharmaceutical actives can be solubilized. The addition of organic carbonates to DBS is critical, since without the carbonates, the pharmaceutical actives are not soluble in DBS. This result is novel and previously unsuggested. It meets the long felt need for non sticky clear sticks containing topically active pharmaceuticals. The composition may also include compounds used in topically applied products such as emollients, penetration enhancers, colorants, perfumes, and the like.

### DEFINITIONS

“Pharmaceutical Active” means a non-antiperspirant compound which has biological activity when applied topically. The biological activity can act either systemically or locally or both

.“Antiperspirant”; or “Antiperspirant Compound” means a compound or combination of compounds recognized by the United States Food and Drug Administration (FDA) as an approved antiperspirant active ingredient. A list of such compounds can be found in the U.S. Federal Register Vol. 68 No. 110 dated Monday Jun. 9, 2003, page 34287. Briefly, these compounds can be described as Aluminum Chloride, several Aluminum Chlorohydrate compounds, several Aluminum Chlorohydrex compounds, several Aluminum Chlorohydrex-polyethylene compounds, Aluminum Sulfate buffered, several Aluminum Zirconium Chlorohydrate compounds, several Aluminum Zirconium Chlorohydrex Glycine compounds, and approved mixtures thereof.

“Substantially” means that in normal commercially practice there is no practical deviation, that “substantially free” means that there is no attempt to add a component of which the composition is substantially free. Similarly, “consisting substantially of” means that the composition is as recited, however the addition of minor additives which will not change the intended nature of basic property of the composition are to be considered as includable. Examples, though limiting, include colorants or odorants.

### DETAILED DESCRIPTION OF THE INVENTION

The instant invention is directed to a clear, that is to say transparent solid stick composition containing at least one topically active, non-antiperspirant pharmaceutically active agent. The stick is intended to be smooth and not tacky to the touch, but nevertheless abradable on the skin with mild hand pressure to deliver the topically active material to the skin, leaving a non sticky, safe and topically absorbable layer on the skin.

The principal components of these sticks are 1-10% , suitably 3-8% (by weight of the total composition) of Dibenzylidene sorbitol, an effective amount of pharmaceutical active and an organic carbonate. The amount of organic carbonate will depend on the amount of pharmaceutically active component pharmacologically desired and permissible by appropriate regulation as well as being soluble in the final composition with out negatively affecting the touch feel and transparency of the final product. Thus the amount of the organic carbonate will usually lie in the range of from about 2 to about 97%, suitably about 85 to about 95% by weight of the total composition. This range is to be considered as indicative and preferred but not limiting.

The organic carbonate is selected from propylene carbonate or butylene carbonate or combinations thereof. The pharmaceutical actives are selected from the group that has biological activity when applied topically to the skin of humans or mammals. Preferred pharmaceutical actives include salicylic acid, retinoic acid, acetyl salicylic acid, and ibuprofen.

It is foreseen that that other compounds that can be included in the composition are those that are well know in the skin care art. Non-limiting examples include esters, oils, moisturizers, gums, colorants, fragrances, silicones, silicas, pH buffers, petrolatums, clays, and surfactants.

### EXAMPLES

### Example 1

### Ibuprofen Stick

| Ingredient | % by weight |
|---|---|
| Dibenzylidene sorbitol | 5.00 |
| Propylene Carbonate | 88.00 |
| Ibuprofen | 5.00 |
| Fumed Silica (Cabosil M5) | 2.00 |
| Total | 100.00 |

The propylene carbonate is slowly heated to 75-80° C. The dibenzylidene sorbitol added slowly and mixed until dissolved. Then the Ibuprofen is added and mixed until dissolved. Thereafter the fumed silica is added and mixed until uniform. The product is poured into containers of the desired shape at 70-75° C. and allowed to cool and set. When cool, the product is ready for use.

### Example 2

### Salicylic Acid Stick

| Ingredient | % by weight |
|---|---|
| Dibenzylidene sorbitol | 3.75 |
| Propylene Carbonate | 84.25 |
| Butylene Carbonate | 10.00 |
| Salicylic Acid | 2.00 |
| Total | 100.00 |

The propylene and butylene carbonates are slowly heated to 75-80° C. The dibenzylidene sorbitol added slowly and mixed until dissolved. Then the salicylic acid is added and mixed until dissolved. The product is poured into containers of the desired shape at 70-75° C. and allowed to cool and set. When cool, the product is ready for use.

### Example 2

### Retinoic Acid Stick

| Ingredient | % by weight |
|---|---|
| Dibenzylidene sorbitol | 8.00 |
| Glyceryl Diacetate | 21.95 |
| Butylene Carbonate | 70.00 |
| Retinoic Acid | 0.05 |
| Total | 100.00 |

The glyceryl diacetate and butylene carbonate are slowly heated to 75-80° C. The dibenzylidene sorbitol added slowly and mixed until dissolved. Then the salicylic acid is added and mixed until dissolved. The product is poured into containers of the desired shape at 70-75° C. and allowed to cool and set. When cool, the product is ready for use

We claim:

1. A clear composition, solid at ambient temperatures, substantially free of mono or polyhydric alcohols other than dibenzylidene sorbitol, consisting substantially of:
at least one organic carbonate,
1-10% by weight of dibenzylidene sorbitol, and
a topically effective amount of at least one non-antiperspirant pharmaceutical active soluble in a combination of the foregoing components and
up to the total amount of the composition by weight of the at least one organic carbonate,

2. The composition of claim 1 in which the organic carbonate is propylene carbonate.

3. The composition of claim 1 in which the organic carbonate is butylene carbonate.

4. The composition of claim 1 in which the organic carbonate is a combination of propylene carbonate and butylene carbonate.

5. The composition of claim 1 in which the pharmaceutical active is selected from the group consisting of ibuprofen, salicylic acid, acetyl salicylic acid and retinoic acid.

6. The composition of claim 5 in which the pharmaceutical active is ibuprofen.

7. The composition of claim 5 in which the pharmaceutical active is acetyl salicylic acid.

8. The composition of claim 5 in which the pharmaceutical active is salicylic acid.

9. The composition of claim 5 in which the pharmaceutical active is retinoic acid.

10. A method of making a clear composition, solid at ambient temperatures, substantially free of mono or polyhydric alcohols consisting substantially of:
least one organic carbonate,
1-10% by weight of dibenzylidene sorbitol, and
a topically effective amount of at least one non-antiperspirant pharmaceutically active soluble in a combination of the foregoing components and
up to the total amount of the composition by weight of the at least one organic carbonate comprising the steps of heating the organic carbonate and dissolving the dibenzylidene sorbitol and the pharmaceutically active therein.

11. The method of claim 10 comprising the additional step of pouring the mixture thus produced into a container of predetermined size, and permitting it to cool.
